(19) United States
(12) Patent Application Publication
(10) Pub. No.: US 2026/0365965 A1
(43) Pub. Date: Nov. 19, 2026

(54) SYSTEM AND METHOD TO CHARACTERIZE VARIOUS BIOPHYSICAL PROPERTIES OF BIOLOGICAL CELLS EXPOSED TO MICROGRAVITY CONDITIONS

(71) Applicant: West Virginia University Board of Governors on Behalf of West Virginia University, Morgantown, WV (US)

(72) Inventors: Soumya Keshavamurthy Srivastava, Morgantown, WV (US); Sai Deepika Reddy Yaram, Morgantown, WV (US)

(21) Appl. No.: 19/678,703

(22) Filed: May 15, 2026

Related U.S. Application Data
(60) Provisional application No. 63/806,781, filed on May 16, 2025.

(51) Int. Cl. C12M 1/36; B64G 7/00; C12M 1/34; C12M 1/42

(52) U.S. Cl. CPC C12M 41/48; C12M 35/04; C12M 41/40; C12M 41/46; B64G 7/00

(57) ABSTRACT

Various examples are provided for characterization of various biophysical properties of biological cells exposed to microgravity conditions. In one example, a method includes placing a cell sample into one or more microcentrifuge tubes; rotating, by a clinostat, the one or more microcentrifuge tubes for a period of time; measuring a dielectric signature of the cell sample; determining one or more cell parameters using the measured dielectric signature; and determining one or more cell properties using the one or more cell parameters and the measured dielectric signature. In another example, a clinostat includes a sample carriage configured to hold one or more sample tubes; a stepper motor that can rotate the sample carriage via a shaft to simulate a microgravity condition for the one or more sample tubes; and stepper motor drive circuitry to control rotational speed of the stepper motor during simulation of the microgravity condition.

### CROSS REFERENCE TO RELATED APPLICATIONS

This application claims priority to, and the benefit of, U.S. provisional application entitled “System and Method to Characterize Various Biophysical Properties of Biological Cells Exposed to Microgravity Conditions” having Ser. No. 63/806,781, filed May 16, 2025, which is hereby incorporated by reference in its entirety.

### BACKGROUND

Every living organism on Earth has grown and evolved experiencing an ever-present pull from Earth's gravity. When traveling in space beyond the Earth's gravitational pull, a phenomenon of near weightlessness occurs due to the lack of gravitational force. When an organism is put in near-weightless conditions, also known as microgravity conditions, there are physiological effects observed. Previous studies have shown that microgravity can impact cytoskeletal organization, membrane fluidity, and stress response pathways in microbial and eukaryotic cells, suggesting that gravity plays a more integral role in cellular regulation than previously understood. In the absence of gravitational force, changes in fluid dynamics, mass transport, and mechanical stresses within the cellular environment can influence essential functions such as nutrient uptake, waste removal, and intracellular signaling.

Sending biological samples to space for research investigations is not a readily available option due to several challenges. First, the costs associated with launching biological experiments in space are extremely high, making it financially prohibitive for many researchers. Additionally, access to space missions is limited, and competition for available slots on spacecraft is intense, leading to long waiting periods before experiments can be conducted. Furthermore, the complexity of maintaining optimal environmental conditions for biological samples during space travel adds another layer of complexity. Conducting experiments in a microgravity environment while in space demands specialized equipment and protocols, which are often difficult to access. As a result, researchers often face significant logistical and technical barriers in pursuing research-based studies, making it challenging to gather data directly from space-based experiments.

The behavior of biological cells under constant gravity conditions is well-understood from conventional, terrestrial analysis. Yet, understanding how reduced-gravity conditions alter cellular membrane integrity, morphology and function remains difficult because true microgravity experiments are prohibitively expensive and infrequent, while ground-based simulators (such as clinostats, random positioning machines, etc.) only approximate weightlessness and often induce shear or centrifugal artifacts. Conventional analyses—microscopy, transcriptomics, metabolomics—provide valuable but indirect or end-point measures of cellular stress and adaptation, and existing electrical-property measurements typically rely on oversimplified single-shell models or low-throughput electrode arrays that lack sensitivity to subtle changes in membrane permittivity or conductivity. As a result, quantifying time-dependent dielectric responses in intact eukaryotic cells under simulated microgravity remains challenging due to limitations in simulation fidelity, measurement resolution, model complexity and statistical robustness.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a plot illustrating an example of the real part of the Clausius-Mossotti factor, Re[K(w)] for yeast cells (Saccharomyces Cerevisiae) at different time frames under simulated microgravity (SMG) environment at a medium conductivity of 0.03 S/m, in accordance with various embodiments of the present disclosure.

FIGS. 2A and 2B illustrate examples of error analysis for specific membrane conductance (FIG. 2A) and capacitance (FIG. 2B), in accordance with various embodiments of the present disclosure.

FIGS. 3A and 3B are schematic representations illustrating examples of cell behavior in a 3DEP microwell under non-uniform electric fields, in accordance with various embodiments of the present disclosure.

FIG. 4 illustrates an example of a dielectric modeling of yeast cells showing transition from double-shell to single-shell approximation, in accordance with various embodiments of the present disclosure.

FIGS. 5A-5F illustrate an example of a stepwise methodology for yeast cell sample preparation and exposure in simulated microgravity using a 3D clinostat, in accordance with various embodiments of the present disclosure.

FIGS. 6A-6E illustrate an example of a workflow for dielectric characterization of yeast cells using the 3DEP analyzer, in accordance with various embodiments of the present disclosure.

FIGS. 7A and 7B illustrate an example of a 2D clinostat setup for simulated microgravity exposure of samples with a controlled rotation system, in accordance with various embodiments of the present disclosure.

FIGS. 7C-7E illustrate an example of a 3D clinostat setup for simulated microgravity exposure of samples with a controlled rotation system, in accordance with various embodiments of the present disclosure.

### DETAILED DESCRIPTION

Disclosed herein are various examples related to characterization of various biophysical properties of biological cells exposed to microgravity conditions. Reference will now be made in detail to the description of the embodiments as illustrated in the drawings, wherein like reference numbers indicate like parts throughout the several views.

Microgravity can alter biological processes, impacting cellular structure, function, and metabolism. In the absence of gravity, cells experience changes that disrupt signal transduction, gene expression, and metabolic pathways, affecting growth rates and cellular viability. Ground-based simulators like clinostats replicate microgravity conditions to study these effects, allowing researchers to examine cellular responses in the lab.

Various examples of the present disclosure use, e.g., Saccharomyces cerevisiae to explore microgravity's impact on yeast metabolism and properties. Yeast cells are exposed to simulated microgravity via a 2D-clinostat and analyzed using dielectrophoresis over 1 to 24 hours. A double-shell model reveals significant morphological and membrane changes under these conditions. Results indicate notable differences in membrane permittivity and conductivity, with microgravity reducing the folding factor in yeast cells, impairing nutrient uptake and energy production. The present disclosure can enhance the understanding of microgravity's effects on eukaryotic cells and contributes to the field of gravitational biology.

### Introduction

Every living organism on Earth has grown and evolved experiencing an ever-present pull from Earth's gravity. When traveling in space beyond the Earth's gravitational pull, a phenomenon of near weightlessness occurs due to the lack of gravitational force. When an organism is put in near-weightless conditions, also known as microgravity conditions, there are physiological effects observed. Yeast cells, like all organisms on Earth, have adapted to function under constant gravitational forces, which influence their growth, division, and overall physiology. When these cells experience microgravity, their intracellular processes can be disrupted, leading to alterations in gene expression, metabolism, and structural integrity. Previous studies have shown that microgravity can impact cytoskeletal organization, membrane fluidity, and stress response pathways in microbial and eukaryotic cells, suggesting that gravity plays a more integral role in cellular regulation than previously understood. In the absence of gravitational force, changes in fluid dynamics, mass transport, and mechanical stresses within the cellular environment can influence essential functions such as nutrient uptake, waste removal, and intracellular signaling. For single-celled organisms like Saccharomyces cerevisiae, which serve as valuable eukaryotic models, these alterations may have cascading effects on cellular efficiency and adaptation. In particular, yeast cells display changes in morphology, including the size ratio of daughter to mother cells, bud direction, and reduced invasive growth. Investigating how yeast cells respond to microgravity provides important insights into the fundamental biological mechanisms governing life in altered gravitational conditions. Prior research has utilized yeast cells in a microgravity environment to study aging effects that can be correlated to humans in space. However, the impact on yeast cell dielectric properties has not been investigated, yet. The present disclosure can not only contribute to the broader field of gravitational biology but can also have potential applications in biotechnology, synthetic biology, and the development of space-based biomanufacturing systems.

Sending biological samples to space for research investigations is not a readily available option due to several challenges. First, the costs associated with launching biological experiments in space are extremely high, making it financially prohibitive for many researchers. Additionally, access to space missions is limited, and competition for available slots on spacecraft is intense, leading to long waiting periods before experiments can be conducted. Furthermore, the complexity of maintaining optimal environmental conditions for biological samples during space travel adds another layer of complexity. Conducting experiments in a microgravity environment while in space demands specialized equipment and protocols, which are often difficult to access. As a result, researchers often face significant logistical and technical barriers in pursuing research-based studies, making it challenging to gather data directly from space-based experiments.

However, microgravity conditions felt in space can be simulated in a ground-based setting through a clinostat. Since Earth's gravity cannot be eliminated, it is rather modified to mimic the effects of microgravity. This is done in a clinostat by rotating it about one or more axes to create a zero-gravity experience for the biological specimen. The clinostat simulation method has been used in other studies to explore the degenerative effects of microgravity on bone cell density and cultured skin cell thickness. While clinostat-based simulations provide valuable insights into how cells respond to microgravity, a deeper understanding of their physiological state requires precise quantitative techniques. Many of these techniques rely on fluorescent labels or costly sample preparation methods, which may inadvertently alter the phenotype or genotype of the cells. To avoid such interference, various embodiments of the present disclosure employ dielectrophoresis, a subtle electrical manipulation technique, to capture the bioelectric signatures of bioparticles without altering their natural state. Dielectrophoresis (DEP) has been applied to characterize, enrich, and sort targeted cells ranging from DNA to mammalian cells.

To quantify the cellular-level changes of yeast cells exposed to simulated microgravity conditions, dielectric signatures can be obtained using a dielectrophoretic crossover technique that analyzes the behavior of cells exposed to non-uniform electric fields. Dielectrophoresis (DEP) is observed when polarizable particles are exposed to a non-uniform electric field with direct current (DC) and alternating current (AC). The polarizability of the particle in relation to the medium it is suspended in determines how the particle behaves in DEP. Using this technique, the dielectric properties of the particle, in this case, a yeast cell, can be determined. The exploration of the specific dielectric properties of cells has been used to define cell health, physiological state, differentiation, function, and intracellular interactions. The specific dielectric properties that correlate with the cellular biological characteristics are membrane conductance, membrane capacitance, cytoplasmic conductivity, and folding factor.

In various embodiments of the present disclosure, Saccharomyces cerevisiae (brewer's yeast) is used as a model organism to explore the effects of microgravity on morphological and cytoplasmic cellular properties. Discovering how microgravity affects cellular structure, and its integrity, promotes a deeper understanding of biological processes in space. Employing a 2D clinostat for microgravity simulation and DEP for characterizing cellular properties demonstrates the extent of changes in response to microgravity exposure. Discovering how cellular structures and properties are altered in response to microgravity environments is important for preventing physiological issues in astronauts and understanding why they may happen.

### Results

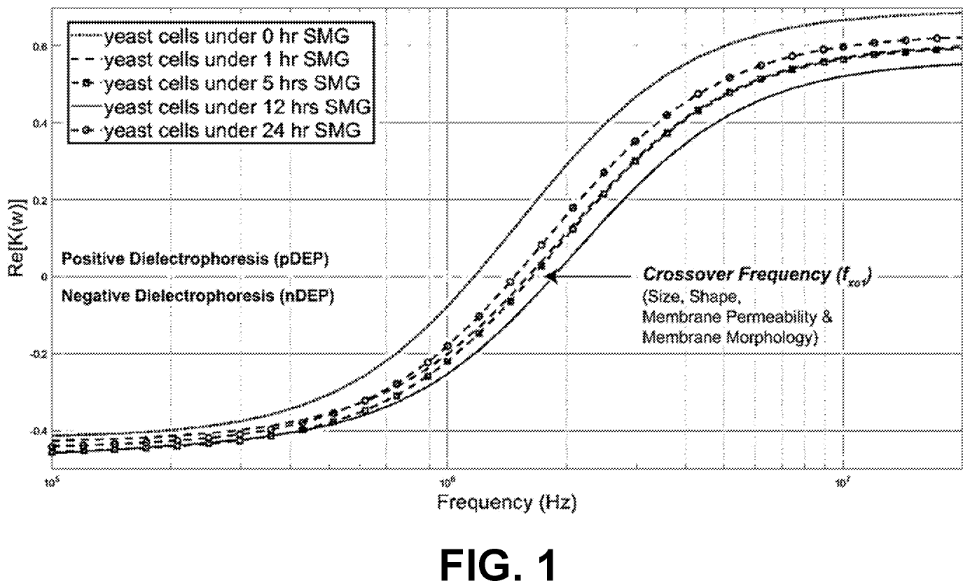

In various embodiments of the present disclosure, brewer's yeast was utilized to simulate the effects of microgravity. Yeast cells were dielectrically modeled using a double-shell model since they have a nucleus, cell membrane, and cell wall to obtain the dielectric properties. The addition of the vacuole compartment in the model was not used since it had a modest impact on the Re[K_{CM}] parameter, which was most visible in the GHz range. Given that the measurements were taken in the MHz range, it was thought permissible to ignore vacuole changes because they were regarded to have a minor impact. The samples obtained after the microgravity exposure, were characterized and analyzed within 20-30 min using the 3DEP dielectrophoretic analyzer (DepTech, Uckfield, U.K.). The experiment was repeated until 10 total runs were obtained for each biological sample with R>0.9 to obtain the statistically significant dielectric properties. The dielectric measurements were obtained in two separate batches for the same biological replicate to ensure reproducibility, i.e., 5 technical replicates were obtained from each biological sample that was sampled as two samples (n=10). The results were statistically consistent and analyzed using GraphPad Prism 10.2.3. Calculating the dielectric properties of the yeast cells using a double-shell model is generally complex, so the curve fit method described in the following sections was used to optimize the values of conductivity and permittivity of all layers reported in Table 1. The Electronic Supplementary Information (ESI) analyzed the influence of the six unknown cell dielectric parameters of yeast cells on Re[K_{CM}]. It was concluded that the cell wall conductivity (σ₁) and the membrane permittivity (ε₂) have greater influence over the Re[K_{CM}] in the frequency ranges of 100 kHz-1 MHz whereas cell membrane conductivity (σ₂) has significant influence at the full frequency range. These three parameters were obtained by curve fitting to the measured data and optimal measurements. MATLAB was used to solve the iterative fitting of the double-shell model (Eqns. 2 and 5 described in the theory section) using data from Table 4 and plotting the Re[K_{CM}] factor as shown in FIG. 1. The cell properties can be related to the crossover frequency using the Clausius-Mossotti factor plot. The first crossover frequency (fₓₒ₁) describes the size, shape, membrane permeability, and membrane morphology of the cell, whereas the second crossover frequency (fₓₒ₂) corresponds to the nucleus-to-cytoplasm volume ratio, providing insights into the relative proportions of these cellular components. The pDEP bandwidth describes the cytoplasm conductivity and nucleus permittivity. In a double-shell model, in cells like E. coli, a secondary crossover frequency (fₓₒ₂) often occurs at rather high frequencies, ranging from 55 to 110 MHz across varied medium conductivities. It was expected that for yeast cells, the second crossover frequency would occur at ultra-high frequencies. Due to the limitations of the equipment, obtaining second crossover frequency was not achievable in this research. As shown in FIG. 1, significant changes were observed in the first crossover frequency (fₓₒ₁) at different time frames, particularly for 5 and 24 hours, demonstrating changes in the membrane morphology.

The plot of Re[K_{CM}] vs. frequency (Hz) in FIG. 1 shows differences in the first crossover frequency (fₓₒ₁) between the normal cells under earth's gravity (0 h control sample) and microgravity-exposed cells (1-24 h). The legend in the plot represents yeast cells exposed to different durations of simulated microgravity (SMG). The lines and markers correspond to: dotted for 0 hours, dashed for 1 hour, squares for 5 hours, solid line for 12 hours, and dashed circles for 24 hour. The crossover frequency, shown at the zero line in the plot that depends on cell size, shape, membrane permeability, and morphology. Positive dielectrophoresis (pDEP) pulls cells toward high-field regions, while negative dielectrophoresis (nDEP) pushes them away. These effects are represented in the plot, helping to understand the cells' electrical properties.

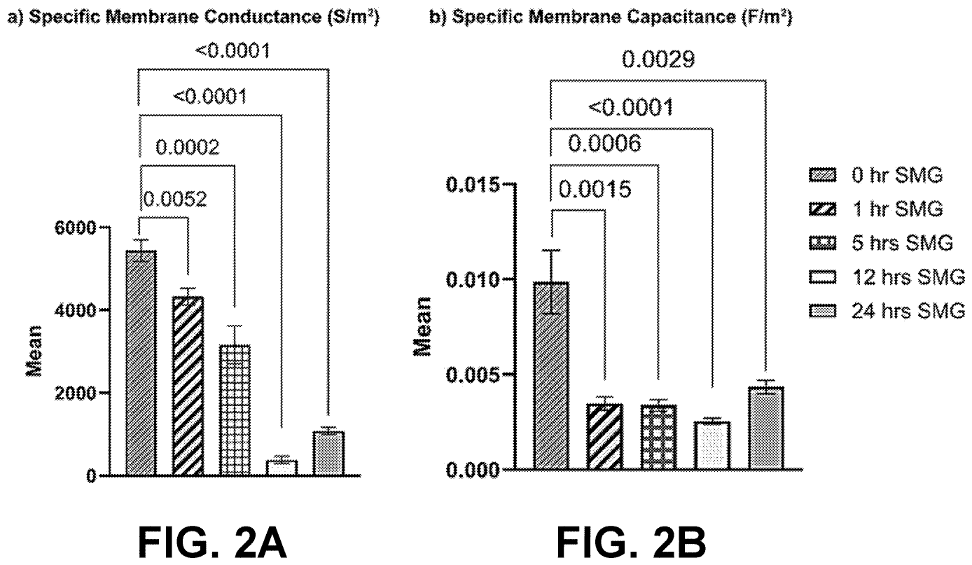

Statistical analysis was conducted, and results are demonstrated in FIGS. 2A and 2B. Error analysis revealed statistically significant differences in (a) specific membrane conductance (FIG. 2A) and (b) specific membrane capacitance (FIG. 2B) across five different time frames, with a sample size of n=10 for all data sets. The Standard Error of the Mean (SEM) is represented as bars, while the histogram displays the mean. The p-value comparing each time frame to the control is shown, with a significance level of α=0.05. The mean of specific membrane conductance and capacitance values, along with associated error bars and p-values, are shown for all the time frames of microgravity exposure including the control sample (0 h). The analyses reveal that membrane conductance experiences a consistent decrease with prolonged exposure to microgravity, with a notably significant difference observed between normal and 24-hour runs. In contrast, membrane capacitance exhibits fluctuations, initially decreasing but subsequently increasing after 12-hour runs. The permeabilization of the cell membrane allows extracellular molecules to enter the cytoplasm. A decrease in membrane capacitance is observed, indicating that cell permeabilization is reduced. A decrease in specific membrane capacitance is linked to cell differentiation and apoptosis. The composition of the membrane is altered by differentiation, resulting in increased thickness and reduced capacitance. In apoptosis, changes to the membrane, such as blebbing and rigidity, are associated with reduced capacitance, signaling a loss of integrity and function. This analytical approach underscores the dynamic nature of cellular responses to microgravity exposure, highlighting distinct patterns in membrane conductance and capacitance changes over time.

### Discussion

Various embodiments of the present disclosure demonstrate the feasibility of utilizing Dielectrophoresis (DEP) to investigate cellular responses to simulated microgravity, presenting a novel approach for elucidating the dielectric signatures of yeast cells under such conditions. Consistent and statistically significant results were obtained through exposure to simulated microgravity across varying timeframes, ranging from 1 to 24 hours, followed by the analysis of dielectric parameters within a short timeframe of 20 to 30 minutes. The double-shell dielectric model was employed to estimate yeast cell dielectric properties, acknowledging the complexity of this approach due to multiple unknowns. To address this challenge, a curve-fitting method was utilized to optimize parameters influencing the Re[K_{CM}] factor plot, facilitating a more precise understanding of cellular responses.

Statistical analyses conducted using GraphPad Prism 10.2.3 revealed significant differences in cell dielectric signatures across different durations of microgravity exposure, as shown in FIGS. 2A and 2B, with pronounced effects observed at prolonged exposure times. The first crossover frequency corresponds to cell size, shape, membrane permeability, and morphology, while the second crossover frequency relates to the nucleus-to-cytoplasm volume ratio. Additionally, the pDEP bandwidth reflects cytoplasmic conductivity and nucleus permittivity. These findings highlight the effectiveness of DEP, particularly when combined with crossover measurement techniques, in characterizing intracellular variations without compromising cell integrity or viability, thereby providing valuable insights into cellular behavior under microgravity conditions.

Furthermore, the cost-effectiveness and accessibility of DEP and clinostat techniques were emphasized in studying cellular properties, in contrast to the logistical challenges associated with space experiments. By simulating microgravity on Earth, researchers are able to efficiently and accurately examine its effects on cellular behavior, thereby enhancing the understanding of fundamental biological processes in space environments. Additionally, by incorporating the rotational dynamics of three independent axes, the use of 3D clinostats provided a more accurate representation of microgravity effects, further advancing the understanding of cellular responses in space environments.

Beyond validating the hypothesis related to microgravity conditions, various embodiments of the present disclosure also identify a gap in research concerning double-shell model analysis. The findings demonstrate the feasibility of efficiently conducting double-shell model analysis through sensitivity analysis of dielectric parameters, offering a novel approach for studying cellular properties under microgravity-like conditions.

The effects of microgravity on various biological systems have been widely studied, revealing significant alterations in cellular structure and function. In microgravity conditions, yeast cells experience changes in metabolism, membrane integrity, and overall cellular physiology, which can serve as a model for understanding broader biological responses to altered gravitational forces. Studying yeast cells under simulated microgravity provides valuable insights into cellular adaptation, differentiation, and apoptosis, helping to uncover fundamental mechanisms that may also be relevant to higher organisms.

This research can be extended to pathogenic yeast species such as Candida auris, which poses a significant risk in immunocompromised environments. Candida species become pathogenic when the host's immune response is weakened, either locally or systemically, with key risk factors including neutropenia, neutrophil dysfunction, and compromised mucosal barriers. With the projected increase in space travel, understanding how microgravity influences yeast cell behavior could aid in detecting and managing fungal infections in space. The significant changes in yeast cell parameters observed within just 12 hours highlight the profound effects that long-term spaceflight could have on microbial behavior and human biology.

As space missions become longer in duration, astronauts are exposed to the challenges of microgravity for extended periods. Astronauts who have been in space for long periods of time face physiological stressors that NASA continues to monitor to mitigate potential health risks. These findings emphasize the importance of continuous health assessments and countermeasures in space environments, ensuring the well-being of astronauts during prolonged missions.

### Methods

### Dielectrophoresis

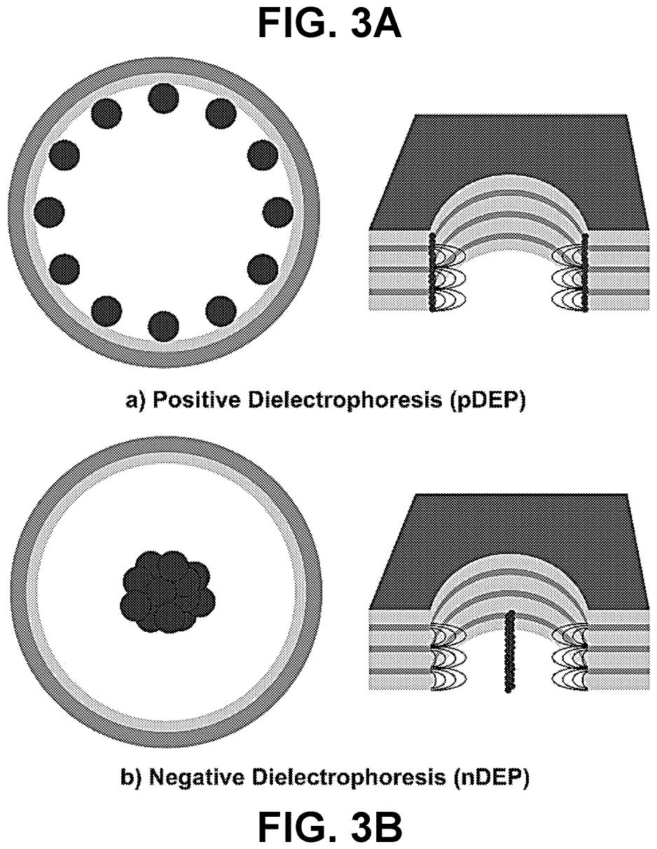

Dielectrophoresis (DEP) is a phenomenon that occurs when polarizable bioparticles, such as cells or microorganisms, are subjected to a non-uniform electric field. DEP is governed by the interaction between the electric field and the inherent electric properties of the particles, primarily their permittivity and conductivity. When a bioparticle, such as a cell, is placed in a microfluidic well, as shown in FIGS. 3A and 3B, its movement will follow one of two directions based on its polarizability relative to the surrounding medium. If cells are more polarizable than the medium, they migrate towards the electrodes (walls in this research), where the electric field is strongest, a phenomenon known as positive dielectrophoresis (pDEP). Conversely, if the opposite is true, where the cells are less polarizable than the medium, they will migrate away from the electrodes, termed negative dielectrophoresis (nDEP). As the transition occurs between nDEP and pDEP or vice versa, cells encounter a specific frequency known as the “crossover frequency” at which they do not experience any DEP force (zero DEP force). FIGS. 3A and 3B show cell behavior under non-uniform electric field in the microwell of 3DEP dielectrophoretic analyzer (DepTech, Uckfield, U.K.). FIG. 3A shows positive dielectrophoresis (pDEP) where the cells are attracted to the walls (high electric field region) and FIG. 3B shows negative dielectrophoresis (nDEP) where the cells move towards the center (low electric field region) in a 3DEP microwell with gold-plated copper electrode along with the electric field lines originating at the walls of the microwell.

The behavior of cells is primarily influenced by the electrical properties of the cell's membrane and cytoplasm, its morphology (including size and shape), and the conductivity of the surrounding medium that the cells are suspended in. The overall magnitude and direction of the DEP force are determined by the relative polarizabilities of the cell and the properties of the surrounding medium. Theoretically, the time-average DEP acting on a spherical cell is given as:

F_{DEP}=2πεₘr³Re[K_{CM}](∇E²) (Equation 1)

where εₘ is the absolute permittivity (εₘ=εᵣεₒ) of the surrounding medium, εᵣ is the relative permittivity, εₒ is the permittivity of the free space, r is the particle radius, Re[K_{CM}] is the real part of the Clausius-Mossotti factor related to the effective polarizability of the particle, E is the amplitude (rms) of the electric field, and ∇ represents the gradient operator. Re[K_{CM}] defines the effective polarizability of the cell as:

$$Re [ {K}_{C M} ] = \frac{( {ε}_{p}^{*} - {ε}_{m}^{*} )}{( {ε}_{p}^{*} + 2 {ε}_{m}^{*} )} \qquad ( Equation 2 )$$

where εₚ* is the complex permittivity of the particle, and εₘ* is the complex permittivity of the media. The complex permittivity ε* is given by:

$${ε}_{p}^{*} = {ε}_{m} - \frac{j {σ}_{p}}{ω} \qquad ( Equation 3 )$$

$${ε}_{p}^{*} = {σ}_{p} + j ω {ε}_{o} {ε}_{p} \qquad ( Equation 4 )$$

where εₚ* is the complex permittivity of the particle, σₚ* is the complex conductivity of the particle, j indicates the imaginary number (j=\sqrt{−1}), and ω is the angular frequency (ω=2πf) of the applied AC field.

Re[K_{CM}] ranges from −0.5 to 1, where positive values show positive dielectrophoresis, pDEP, and negative values of Re[K_{CM}] denote negative dielectrophoresis, nDEP.

### Dielectric Double Shell Model

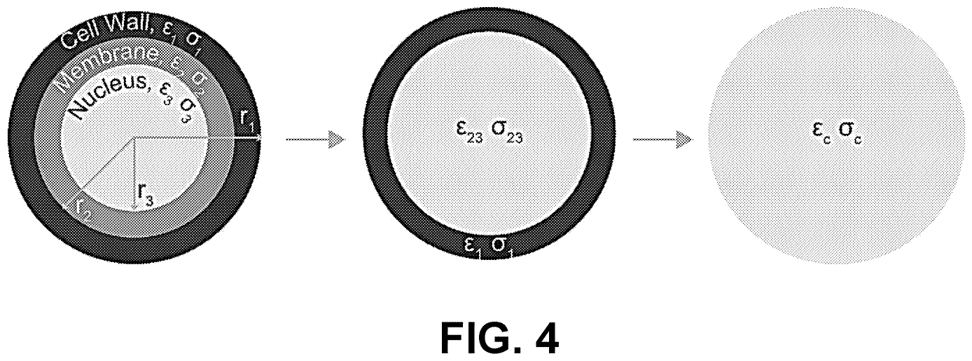

A simple dielectric model, known as the ‘single shell’ model, accurately models the dielectric behavior of cells that lack internal organelles, especially, the nucleus, but do possess a plasma membrane and not the cell wall. However, yeast cells are enclosed by a cell wall and contain internal organelles such as the vacuole, nucleus, and mitochondria, making them more complex to model using a single shell. Here, the double shell model is utilized to obtain the dielectric parameters, i.e., the Clausius Mossotti (CM) factor (in Eqn. 2) and yeast cells are simplified to mimic dielectric spheres, as shown in FIG. 4. FIG. 4 is a pictorial representation of the dielectric double-shell model for yeast cells, which includes internal components such as the cell wall, vacuole, nucleus, and mitochondria. To simplify the analysis, a single-shell model is derived to approximate effective cell permittivity and conductivity. While the single-shell model is suitable for cells lacking internal organelles, the double-shell model is employed here to more accurately represent the complex internal structure of yeast. The resulting Clausius-Mossotti (CM) factor is used to calculate dielectric properties, with yeast cells approximated as dielectric spheres.

Due to the complex nature of the model, the effective permittivity of the cell (ε_{c}′*) is determined through an iterative process. Initially, the nucleus and its surrounding envelope, constituting the innermost layer of the cell, are approximated and substituted with a sphere of equivalent size (nucleus radius plus the radius of the cell membrane). The effective permittivity is then defined with the complex permittivity of the nucleus (εₙᵤ*) and the cell membrane (εₙᵤₘₑₘ*), where ‘a’ represents the ratio of the external (cell membrane) radius to the internal (nucleus) radius of the combined layers.

$${ε}_{nucmem}^{′ *} = {ε}_{nucmem} \frac{{( a )}^{3} + 2 ( \frac{{ε}_{nu}^{*} - {ε}_{numem}^{*}}{{ε}_{nu}^{*} + 2 {ε}_{numem}^{*}} )}{{( a )}^{3} - ( \frac{{ε}_{nu}^{*} - {ε}_{numem}^{*}}{{ε}_{nu}^{*} + 2 {ε}_{numem}^{*}} )} \qquad ( Equation 5 )$$

Subsequently, the iterative process continues until the entire cell is simplified to be a single sphere, encompassing the double-layer spherical model comprising the nucleus, cell membrane, and cell wall. This approach combines the effective permittivity of the nucleus and cell membrane, as described in Eqn. 5, with the surrounding cell wall to derive the effective cell permittivity. The effective values of the components are then employed in Eqn. 2. The parameters yielding the best fit to the data are utilized to obtain the dielectric properties of the cell components.

### Obtaining Dielectric Cell Parameters Using a Double-Shell Model

Various embodiments of the present disclosure utilize a double-shell model to analyze yeast cells, incorporating three geometric parameters (cell radius, nucleus radius, and membrane thickness) and six dielectric parameters (permittivity and conductivity for each compartment-cell wall, cell membrane, and cytoplasm). These parameters are determined through curve fitting to the measured data i.e., plotting Re[K_{CM}] vs. frequency data. It is complex and cumbersome to extract all the characteristics as some parameters are frequency-independent and some parameters are sensitive over the frequency range of collected data. To overcome this, a sensitivity analysis needs to identify the parameters that have the greatest influence on the Re[K_{CM}] spectrum across the experimental data frequency range. The geometric parameters of yeast cells that will be used to model the individual compartments obtained from the literature are summarized in Table 2.

The parameter values chosen for optimization, determined through sensitivity analysis, are derived by fitting curves to the measured Re[K_{CM}] spectra. The Nelder-Mead simplex optimization method (modeled using Python-COLAB) is used to minimize the error function:

$$E = \sqrt{{\int }_{{f}_{min}}^{{f}_{max}} {[ {K}_{meas} ( f , {x}_{1} , … , {x}_{n} ) - {K}_{opt} ( f , {x}_{1} , … , {x}_{n} ) ]}^{2} df} \qquad ( Equation 6 )$$

where Kₘₑₐₛ(f,x₁, . . . ,xₙ) represents the measured spectra and Kₒₚₜ(f,x₁, . . . ,xₙ) indicates the fitted Re[K_{CM}] spectra.

A sensitivity analysis of the dielectric parameters in the two-shell model was conducted to identify the most significant variables for data fitting and to determine which parameters remain constant when comparing experimental data for yeast cells at different time points. The results from prior studies revealed that the cell wall's permittivity has minimal impact on Re[K_{CM}] between 100 KHz and 1 MHz, while its conductivity significantly affects Re[K_{CM}]. Conversely, the cell membrane's permittivity notably influences Re[K_{CM}] within the 100 kHz to 1 MHz range, whereas its conductivity affects the entire frequency spectrum. Both the permittivity and conductivity of the cytoplasm have negligible effects on Re[K_{CM}] between 100 KHz and 1 MHz. Thus, cytoplasm conductivity and permittivity and the cell wall's permittivity are maintained constant.

### DEP Suspending Medium and Sample Preparation

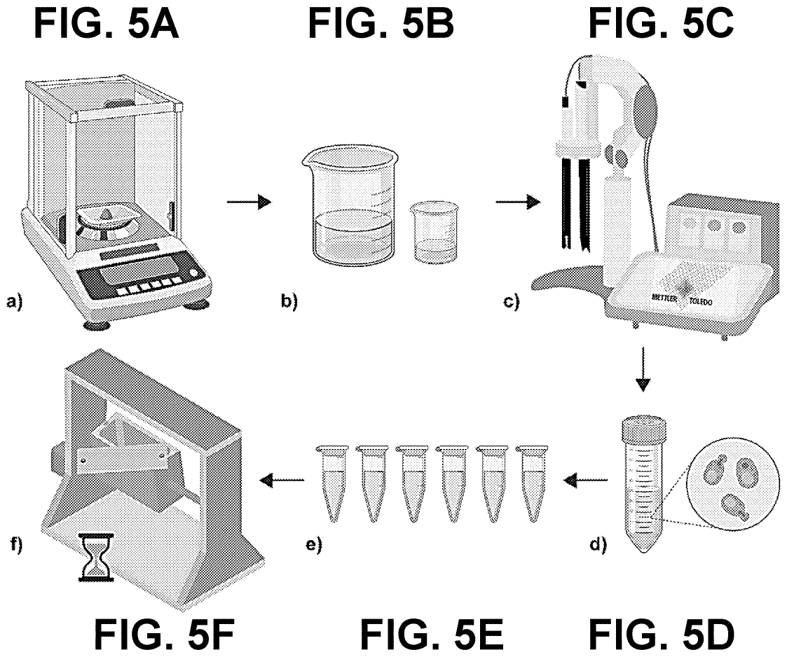

Prior to conducting dielectric measurements and analysis, a suspending media (buffer) for the yeast cells was prepared by dissolving 1.25 g of D-glucose in 25 mL of deionized (DI) water. The medium conductivity measured using the Mettler Toledo multiparameter instrument was adjusted to 100 μS/m by dissolving 0.09 g of sodium chloride in 10 mL of DI water, as illustrated in FIGS. 5A-5F. FIGS. 5A-5F show the methodology involved in sample preparation for simulated microgravity experiment, including a) Weighed 1.25 g of D-Glucose and 0.09 g of NaCl (FIG. 5A), b) Added D-Glucose to 25 ml of DI water and NaCl to 10 ml of DI water (FIG. 5B), c) Adjusted the media conductivity to 100 μS/m using NaCl solution (FIG. 5C), d) Added yeast to the suspending media (FIG. 5D), e) Aliquoted into six microcentrifuge tubes (FIG. 5E), and f) Rotated the yeast cell sample for certain chosen time point (FIG. 5F) and the same steps were repeated for other time frames (hour glass represents the time point rotations in the clinostat).

Yeast cells (dry active yeast, Kroger) were sparingly added to approximately 10 mL of the buffer solution. Once the concentration was optimized through visualization under a microscope, the yeast sample was aliquoted into six microcentrifuge tubes, 1 ml each, and balanced to spin within a 2D clinostat. The clinostat was set to spin at various time points for 1, 5, 12, and 24 hours, depending on the time of microgravity exposure for each trial. Control sample trials were conducted at 0 hours without microgravity exposure, under Earth's gravity conditions.

### Dielectric Characterization Experiments

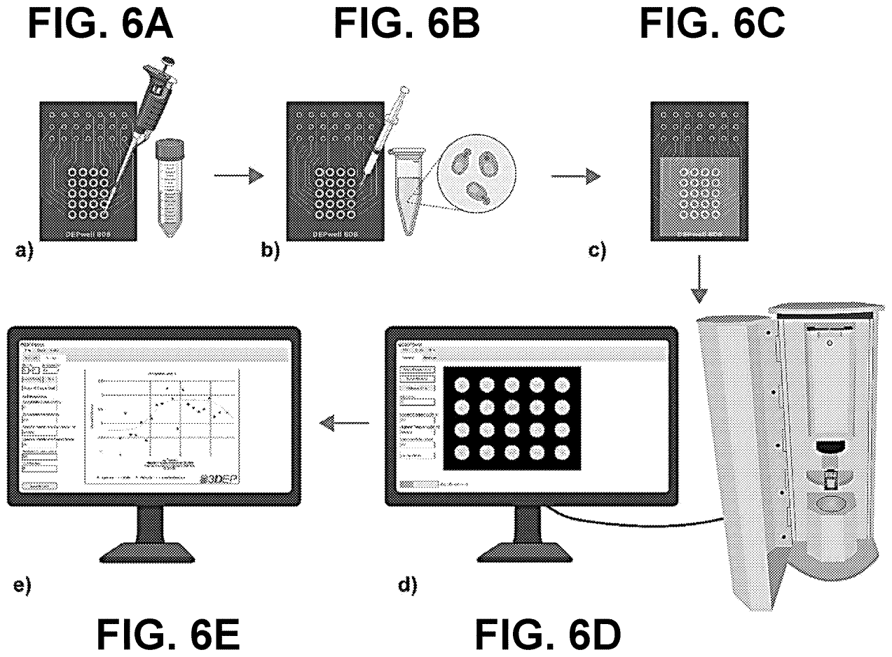

Dielectric characterizations or dielectric signatures were obtained using the 3DEP analyzer and a DEPwell 806 chip (DepTech, Uckfield, U.K.). A chip containing cell suspensions is inserted into a specially made zero-insertion force mount and connected to AC signal generators, as shown in FIGS. 6A-6E. A CMOS camera with bi-telecentric optics is used to monitor and illuminate the chip, allowing for the simultaneous monitoring of all the 20 wells. The wells are powered by 20 user-assigned frequencies, spanning from 1 kHz to 45 MHz, during the experiment. An image analysis algorithm tracks the mobility of the particles, and each well's concentric ring's light absorption is measured. To compute the Clausius-Mossotti factor, which represents the dielectric characteristics of the cells, variations in light intensity are utilized.

The 20-microwell chip is first washed with the DEP suspending medium, i.e., D-glucose buffer. Then, approximately 100 μL of yeast cell suspension is added to the chip using a 1.5 ml syringe with a 25G Bent and blunt needle. A glass coverslip is placed over the chip to prevent air bubbles from forming within the wells. The sample-loaded chip is then inserted into the 3DEP analyzer, where cell behavior is analyzed at 20 Vₚₚ with frequencies swept between 0.5 kHz and 45 MHz for 30 seconds. Prior to starting the experiment, the medium conductivity is set to 100 μS/m, and the cell radius is adjusted to 3 μm within the 3DEP software. A detailed illustration of the methodology involved in using 3DEP dielectrophoretic analyzer (DepTech, Uckfield, U.K.) for dielectric characterization is shown in FIGS. 6A-6E, including a) washed the 3DEP chip with the suspending medium, i.e., D-Glucose buffer (FIG. 6A), b) yeast cell suspension loaded using a syringe (FIG. 6B), c) coverslip placed after removing air bubbles (FIG. 6C), d) chip inserted in the 3DEP analyzer to analyze cell behavior at varying frequencies and fixed peak-to-peak voltage (FIG. 6D), and e) results exhibited by 3DEP software after auto-fitting to single shell model at R>0.9 (FIG. 6E).

### 3DEP Analyzer Curve-Fitting Procedure

Dielectrophoresis (DEP) is a powerful technique used to characterize cellular properties by analyzing their movement in response to non-uniform electric fields. By observing how cells migrate under different electrical conditions, DEP allows researchers to assess cellular behavior without the need for fluorescent labels or invasive modifications. When exposed to a non-uniform electric field, cells experience varying force intensities, leading to strong, weak, or negligible forces near the wall electrodes lining the microwells. When frequencies ranging from 0.5 kHz to 45 MHz were applied, cells exhibited negative dielectrophoresis (nDEP), when frequencies were in the kHz range causing them to migrate toward the center of the well, away from the electrodes embedded in the walls. Conversely, at higher frequencies, in the MHz range, the same cells experienced positive dielectrophoresis (pDEP), leading them to move toward the electrodes, closer to the microwell walls. The 3DEP system utilized a collimated light source and an integrated CMOS camera to measure light intensity across the microwells, which was then analyzed to track cell migration patterns and generate the dielectric spectrum by fitting the data points to the Clausius-Mossotti function. Each experiment was repeated for at least 10 technical replicates per biological replicate, with a minimum of three biological replicates, resulting in approximately 30 experimental runs. This ensured accuracy until a Pearson correlation coefficient of at least 0.9 was achieved using the 3DEP Auto-Fit Single Shell Model. A detailed explanation of the operational principles and data analysis for the 3DEP system has been provided in a separate source.

### Statistical Analysis

Statistical analysis was conducted using GraphPad Prism 10.2.3. Data on yeast cells at various time points of microgravity exposure, specifically for specific membrane conductance and capacitance, were compiled in GraphPad Prism 10.2.3 to perform a normality test using the Anderson-Darling test. Since the comparison group (0 hours in both cases) deviated from normality, the p-value was determined using the Mann-Whitney test, a non-parametric t-test.

### Clinostats

The primary purpose of a clinostat is to rotate biological samples along a single horizontal axis. Clinostats can be classified into two-dimensional (2D), rotating about the horizontal axis through a single motor, three-dimensional (3D), rotating about two orthogonal axes through two motors, and random positioning machines (RPMs), where both axes rotate randomly. By maintaining a consistently high rotation speed, these clinostats effectively counteract the settling of particles or specimens due to gravity, creating a condition akin to weightlessness in a limited space. This feature is particularly beneficial for scientists, enabling them to study the effects of a microgravity-like environment on various subjects, including individual cells, and to gain valuable insights into its impacts. To achieve an effective simulation of microgravity, the rotation rate of the sample around a horizontal axis needs to be consistent and sufficiently fast to prevent responses to gravity, yet not so fast as to generate substantial accelerating force.

Various embodiments of the present disclosure employed a 2D clinostat to simulate microgravity-like conditions on yeast cells. Developing and utilizing the clinostat proved to be a cost-effective alternative to sending samples to the International Space Station (ISS). Additionally, it allowed for easier monitoring and enabled multiple experiments to be conducted without the constraints of waiting for the next space mission.

### 2D Clinostat Design

A clinostat can simulate the microgravity effects by distributing the gravitational pull about the axes of the rotation by changing the gravitational vector. The objective is to determine the rotational speed needed to counteract particle sedimentation caused by gravity. It is important to find a balance that prevents excessive centrifugal forces within the flask. These calculations will establish a range of suitable rotational values for motor selection when making a purchase. Initial calculations involved utilizing a red blood cell and a bacterial cell as reference standards, employing the physical properties outlined in Table 3. It is assumed that the density of cell culture media is equal to the density of water and the temperature of a cell culture incubator. The sedimentation speed of the cells, V_{sed}, in media under normal gravity can be calculated using the following:

$${V}_{sed} = ( \frac{2}{9} ) ( {ρ}_{particle} - {ρ}_{fluid} ) ( \frac{g}{μ} ) {a}^{2} \qquad ( Equation 7 )$$

where ρ_{particle} is the density of the particle, ρ_{fluid} is the density of the fluid, g is the acceleration due to gravity, μ is the viscosity of the liquid medium, and a is the effective stroke radius of the particle.

The radius that a particle travels during the clinorotation, r, is directly proportional to the ratio of the diffusion coefficient of the cell in a liquid medium (D_{AB}) and the sedimentation speed (V_sed) calculated using Eqn. 6 yielding:

$$r = \frac{4 π {D}_{A B}}{{V}_{s e d}} \qquad ( Equation 8 )$$

This radius takes into consideration the sedimentation that occurs as the particle moves along a circular path and also accounts for the random dispersion caused by the Brownian motion. The rotation period, τ, is determined using Eqn. 8. This represents the expected time required for the cell to complete a single rotation while accounting for both clinorotation and Brownian motion. In this equation, ‘r’ signifies the radius of the particle's circular path during one rotation. From this derived value, the angular velocity is computed using Eqn. 9, and it is subsequently converted into the minimum RPM (revolutions per minute) as demonstrated in Eqn. 10.

$$τ = \frac{2 π r}{{V}_{s e d}} \qquad ( Equation 9 )$$

$$ω = \frac{2 π}{τ} \qquad ( Equation 10 )$$

$${RPM}_{minimum} = \frac{60 ω}{2 π} \qquad ( Equation 11 )$$

The diffusion coefficient of cells, D_{AB}, is calculated based on liquid-mass diffusion given by:

$${D}_{A B} = \frac{k T}{6 π {r}_{A} {μ}_{B}} \qquad ( Equation 12 )$$

where k is the Boltzmann constant, Tis the absolute temperature, r_{A} is the molecule radius of the cell, and μ_{B} is the viscosity of the solvent.

### 2D Clinostat Fabrication

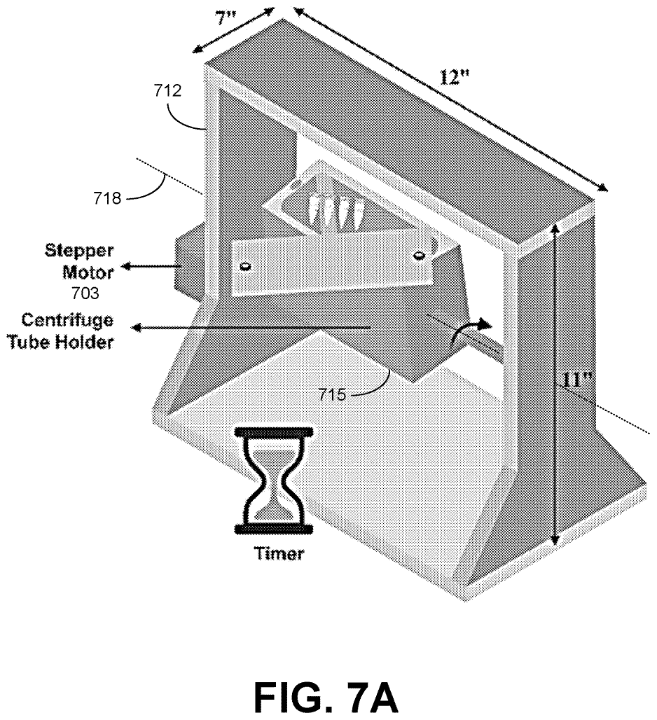

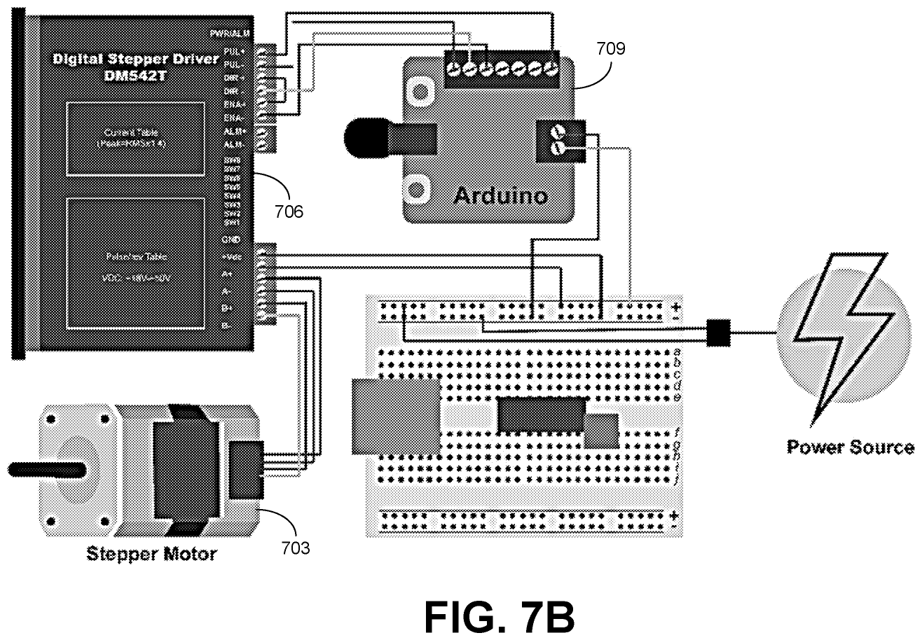

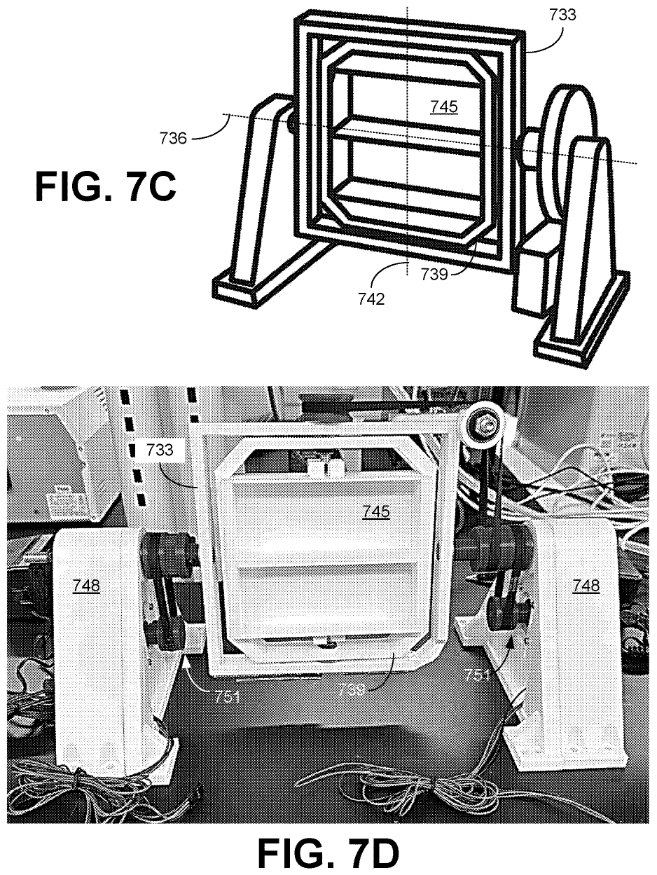

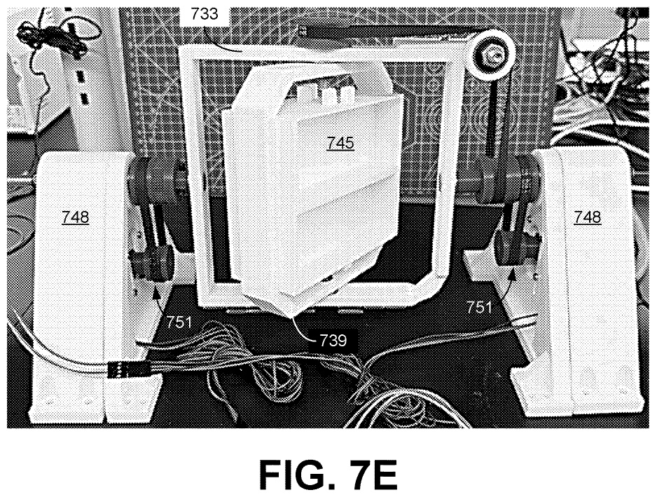

FIGS. 7A and 7B illustrate an example of a 2D clinostat setup for simulated microgravity exposure of samples with a controlled rotation system. The 2D clinostat comprises a stepper motor 703 connected to a Digital Stepper Driver (DM542T) 706, which was controlled via an Arduino programmable microcontroller board 709 (or other programmable processing circuitry including a processor and memory) to regulate the clinostat's rotational speed (rpm). The calculated rotational speed for the horizontal axis of a 2D clinostat is comparable to a true microgravity environment. The clinostat frame 712 was designed to support rotational speeds up to 75 RPM continuously and accommodate the weight of the motor 703 and centrifuge tubes. Solid walls were chosen over frame-like rods for stability. The design of the carriage 715 focused on accommodating centrifuge tubes to simulate microgravity. The finalized design includes triangular legs, bearings, and a rotating system, and measures 12″×7″×11″, striking a balance between size and attachment flexibility. A flask basket, made from a single aluminum piece, can hold four centrifuge tubes securely and features a lid for swift access. Quarter-turn fasteners enable rapid removal, which is important for biological experiments. The carriage 715, which can be milled from aluminum, holds flasks tightly, utilizing quarter-fasteners for the lid and threaded axles for secure assembly to the frame during rotation. Stainless steel axles ensure the carriage's weight-bearing capacity. Aluminum particularly extruded 6061, was chosen for construction due to its cleanability for biological use. Its resistance to bleach matches stainless steel, though it may react slowly with ethanol unless anodized. Autoclaving showed comparable durability to commercial aluminum pressure cookers, affirming aluminum's suitability for the clinostat's cleaning and usage requirements while remaining cost-effective. The 2D clinostat was fabricated using a 3D printer, which can hold 1.5 mL centrifuge tubes and rotate along the x-axis 718 to replicate the microgravity effect. The 2D clinostat has a rotating axis that rotates along the x-axis 718, and the pulley system is designed to control the rotation using the stepper motor as shown in FIG. 7A.

The 2D Clinostat set-up components needed to expose yeast samples to simulated microgravity environment are illustrated in FIGS. 7A and 7B. It includes the 3D-printed 2D clinostat setup, designed to hold four 1.5 mL centrifuge tubes as shown in FIG. 7A and the electrical circuit connections for the system including an Arduino programmable microcontroller board 709, to control the rotational speed (RPM) as shown in FIG. 7B. This setup allows the samples to be rotated at various time intervals to investigate differences in properties and the effects of microgravity. The stepper motor 703 is connected to the Arduino programmable microcontroller board 709 that controls the rpm. The maximum rotational speed investigated for examining the impact of microgravity on cells was approximately 50-60 rotations per minute. Hence, the rotation is maintained at a consistent speed of around 54.4 revolutions per minute (rpm). The cells in the sample are exposed to microgravity and sampled at various time frames, i.e., at 0 h (control), 1 h, 5 h, 12 h, and 24 h.

TABLE 1
Fitted dielectric parameter of yeast
cells (Saccharomyces cerevisiae)

| | Time under simulated microgravity | | | | |
|---|---|---|---|---|---|
| Parameter | environment (hours) | | | | |
| (unit) | 0 (control) | 1 | 5 | 12 | 24 |
| ε₃ (F/m) | 78 | 78 | 78 | 78 | 78 |
| σ₃ (S/m) | 0.2 | 0.2 | 0.2 | 0.2 | 0.2 |
| C₂ (F/m²) | 0.0098 | 0.0034 | 0.0033 | 0.0025 | 0.0043 |
| G₂ (S/m²) | 5440.61 | 4326.14 | 3161.86 | 294.06 | 900.50 |
| ε₁ (F/m) | 60 | 60 | 60 | 60 | 60 |
| σ₁ (S/m) | 3.58 × | 2.92 × | 1.22 × | 6.91 × | 4.35 × |
| | 10⁻⁴ | 10⁻⁴ | 10⁻⁴ | 10⁻⁴ | 10⁻⁴ |
| φ | 1 | 0.37 | 0.36 | 0.27 | 0.47 |
| fₓₒ₁ (kHz) | 1170 | 1590 | 1630 | 1850 | 1480 |

This table presents the dielectric parameters of yeast cells subjected to simulated microgravity for 0 (control), 1, 5, 12, and 24 hours. While the relative permittivities (ε₃ and ε₁) and medium conductivity (σ₃) are kept constant, significant variations are observed in specific membrane capacitance (C₂), specific membrane conductance (G₂), cytoplasm conductivity (σ₁), folding factor (φ), and first crossover frequency (fₓₒ₁). Notably, C₂ and G₂ decrease initially and show partial recovery by 24 hours, while fₓₒ₁ increases over time, indicating progressive cellular dielectric changes under microgravity.

TABLE 2
Geometrical and Dielectric Properties
of Yeast Cell Compartments

| Parameter (units) | Value |
|---|---|
| r₁ (μm) | 3 |
| σ₁ (μS/m) | 140 |
| ε₁ (μF/m) | 60 |
| r₂ (μm) | 2.78 |
| σ₂ (μS/m) | 2.5 × 10⁻³ |
| ε₂ (μF/m) | 6 |
| r₃ (μm) | 2.77 |
| σ₃ (μS/m) | 2000 |
| ε₃ (μF/m) | 50 |

Geometrical and dielectric parameters of yeast cells for each compartment, i.e., cell wall, cell membrane, and cytoplasm, including the radius, r, conductivity, σ, and permittivity, ε.

TABLE 3
Parameters Used to Calculate Rotational
Speed in Simulated Microgravity
Experiments for RBCs, Bacterial
Cells, and Culture Media

| Physical Property | Value |
|---|---|
| Volume, V_{rbc} (cm³) | 8.7 × 10⁻¹¹ |
| Volume, V_{bc} (cm³) | 3.93 × 10⁻¹² |
| Density, ρ_{rbc} (g/cm³) | 1.11 |
| Density, ρ_{bc} (g/cm³) | 1.12 |
| Density, ρ_{ccm} (g/cm³) | 1 |
| Adensity, δ ρ_{rbc-ccm} (g/cm³) | 1.055 |
| Adensity, δ ρ_{bc-ccm} (g/cm³) | 1.058 |
| Length of the cell culture flask (cm) | 4.95 |
| Width of the cell culture flask (cm) | 2.54 |
| Boltzmann constant, k_{b} (cm² g/s² k) | 1.38 × 10⁻¹⁶ |
| Temperature (k) | 310 |
| Rotations per minute (rpm) | 60 |
| Angular velocity, ω (rad/s) | 6.28 |
| Acceleration, a_{centrifugal} (cm/s²) | 195.42 |
| Acceleration due to gravity, g (cm/s²) | 980 |
| Viscosity, μ_{ccm} (pa-s) | 0.00705 |
| D_{bc} (cm²/s) | 2 × 10⁻¹¹ |
| D_{rbc} (cm²/s) | 7.85 × 10⁻¹⁰ |
| Effective stokes radius, a_{bc} (cm) | 7.29 × 10⁻⁶ |
| Effective stokes radius, a_{rbc} (cm) | 3.84 × 10⁻⁵ |

List of properties to calculate the rotational speed using the Eqns. 7-12. The subscripts, rbc, bc, and ccm refer to red blood cells, bacterial cells, and cell culture media respectively.

TABLE 4
Calculated Rotational Parameters for Red Blood Cells
and Bacterial Cells Based on Equations 7-12

| | V_{sediment} | | | ω | RPMₘᵢₙ |
|---|---|---|---|---|---|
| Cells | (cm/s) | r (cm) | τ (s) | (rad/s) | (rev/min) |
| Bacterial Cells | 3.45 × 10⁻⁵ | 7.29 × 10⁻⁶ | 1.33 | 4.73 | 45.16 |
| Red Blood Cells | 2.57 × 10⁻⁴ | 3.84 × 10⁻⁵ | 9.41 × 10⁻¹ | 6.68 | 63.75 |

Calculated parameters obtained for red blood cells and bacterial cells using Eqns. 7-12.

### 3D Clinostat Design

FIGS. 7C-7E illustrate an example of a 3D clinostat setup for simulated microgravity exposure of samples with a controlled rotation system. As with the 2D clinostat, the 3D clinostat can simulate the microgravity effects by distributing the gravitational pull about the axes of the rotation by changing the gravitational vector. The objective is to determine the rotational speed required to counteract particle sedimentation caused by gravity, while preventing excessive centrifugal forces within the flask. The 3D clinostat employs two perpendicular rotational axes to continuously randomize the gravity vector experienced by the sample(s), thereby creating an environment that approximates microgravity.

A low-cost, benchtop 3D clinostat is provided to simulate microgravity conditions for biological and material-science research. Unlike commercially available 3D clinostats and random positioning machines, which are often prohibitively expensive, the proposed design uses affordable, readily available components while maintaining stable and controllable rotation suitable for preliminary studies. The disclosed 3D clinostat can be used by laboratories conducting early-stage microgravity experiments—including cancer research, tissue engineering, stem cell studies, and 3D culture development—as well as by educational laboratories and research groups with limited funding. Researchers can reproduce microgravity-like conditions adequate for spheroid formation and cellular response studies.

FIG. 7C graphically illustrates an example of the 3D clinostat, which comprises an outer frame 733 (which rotates about a first (horizontal) rotational axis 736) and an inner frame 739 (which rotates about a second (vertical) rotational axis 742). A carriage 745 of the sample tube holding platform is located at the center of rotation. Two stepper motors 703 can provide independent control of each of the first and second rotational axis 736 and 742, with corresponding motor drivers 706 for each stepper motor 703. The motor drivers 706 can be mounted on a common control board. Structural components, bearings, and fasteners can be 3D-printed for lightweight assembly. FIGS. 7D and 7E are images of a fabricated 3D clinostat. The two stepper motors 703 are mounted on opposite sides of the support frame 748 and utilize belt drives and pulleys 751 to control rotation of about the first and second rotational axes 736 and 742. A cover for the carriage 745 of the sample tube holding platform is not shown in the image. The frames include 3D-printed components and low-friction ball bearings that are used to ensure smooth rotation. The sample holder accommodates standard cell-culture T-25 flasks and can be further adapted for other cell culture vessels (e.g., 6-well plates, T-flasks, microfluidic devices, conical tubes).

The electronic control system is similar to the 2D clinostat described with respect to FIG. 7B. The 3D clinostat can comprise stepper motors 703 connected to motor drives (e.g., Digital Stepper Driver DM542T) 706, which can be controlled via an Arduino programmable microcontroller board 709 or other programmable processing circuitry (including a processor and memory) to regulate the clinostat's rotational speeds (rpm). In the example of FIGS. 7C-7E, the motion system electronics comprise an Arduino controller, Stepper motors, Stepper motor drivers, pulley belts, adapters, universal breadboards, male-to-male breadboard pins, and connectors. The mechanical components comprise 3D-printed frame parts, bearings, mounting brackets, screws, nuts, and bolts, skateboard bearings, adhesive wheel weights. Control software allows a user to set independent or synchronized rotation speeds (e.g., in a range from about 1-20 rpm, or about 1-15 rpm, or about 1-10 rpm, or other appropriate range), acceleration profiles, and timing intervals. Continuous randomization of the gravity vector can be achieved by varying rotation speeds on the two axes 736 and 742.

The 3D clinostat can be fabricated by 3D printing the outer and inner rotational frames, 733 and 739, and the mounting brackets for the stepper motors 703 and bearings. The structural frame can then be assembled using screws, fasteners, and/or other appropriate fixing devices. Mechanical parts can then be installed. For example, bearings can be inserted into rotation joints and stepper motors 703 can be mounted onto printed brackets. Motor shafts can be attached to each frame 733 and 739 using couplers, belts and pulleys, etc. The carriage 745 of the sample tube holding platform can then be installed in the geometric center as shown in FIG. 7D. The electronic circuitry can be assembled by connecting the Arduino 709 to the motor drivers 706, wiring the motors, power supply, and user interface (if applicable), and uploading rotational control software. Once installed, the system operation can be verified for independent and simultaneous rotation about both axes, adjustment of operational speed within the desired range, confirmation of smooth motion with the absence of vibration.

To use the 3D clinostat, biological samples can be prepared and sealed in standard culture vessels, such as described with respect to the example of FIGS. 5A-5E. The samples can be mounted (and covered) in the carriage 745 of the sample tube holding platform. Rotation speeds for the first and second axes 736 and 742 can be set and initiated (e.g., in a range from about 1-20 rpm, or about 1-15 rpm, or about 1-10 rpm, or other appropriate range). For example, the rotational speed for the inner frame 739 can be set to 5 rpm and for the outer frame 733 can be set to 8 rpm. The system can then be powered up and rotation initiated. Environmental conditions (e.g., temperature, CO₂, etc.) can be maintained by placing the clinostat inside an incubator. The experiment can be run for a predetermined period of time (e.g., 1 hour, 2 hours, 5 hours, 10 hours, 12 hours, 12-72 hours, or other defined period of time depending on the biological goals). At the end of the run period, the rotation is stopped, the sample(s) are retrieved, and the process sample(s) analyzed (e.g., viability assays, imaging, qPCR).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A method for characterizing biophysical properties of cells exposed to microgravity conditions, comprising:
placing a cell sample into one or more microcentrifuge tubes;
rotating, by a clinostat, the one or more microcentrifuge tubes for a period of time;
measuring, by a three-dimensional electrophysiology platform (3DEP) analyzer, a dielectric signature of the cell sample;
determining, by a computing device, one or more cell parameters based at least in part on the measured dielectric signature; and
determining, by the computing device, one or more cell properties based at least in part on the one or more cell parameters and the measured dielectric signature.

2. The method of claim 1, wherein determining one or more cell parameters further comprises:
approximating, by the computing device, a first shell of a cell of the cell sample, the first shell comprising a nucleus and a membrane;
determining, by the computing device, an effective permeability of the first shell;
approximating, by the computing device, a second shell of the cell, the second shell comprising the first shell and a cell wall;
determining, by the computing device, an effective permeability of the second shell;
deriving, by the computing device, an effective cell permittivity based at least in part on the effective permeability of the first shell and the effective permeability of the second shell;
determining, by the computing device, a real part of a Clausius-Mossotti (CM) factor (Re[K_{CM}]) based at least in part on the effective cell permittivity; and
determining the one or more cell parameters based at least in part on the Re[K_{CM}]).

3. The method of claim 1, wherein the measured dielectric signature comprises a measured real part of a Clausius-Mossotti (CM) factor (Re[K_{CM}]), one or more measured crossover frequencies of the cell sample, or a combination of any thereof.

4. The method of claim 1, further comprising preparing the cell sample by:
dissolving D-glucose in deionized (DI) water to form a suspending medium;
adjusting a medium conductivity by dissolving sodium chloride in the suspending medium; and
adding a plurality of cells to the suspending medium.

5. The method of claim 1, wherein measuring the dielectric signature further comprises:
loading the cell sample into a 3DEP chip;
inserting the 3DEP chip into the 3DEP analyzer; and
analyzing, by the 3DEP analyzer, the dielectric signature at a plurality of frequencies and at a fixed voltage.

6. The method of claim 1, wherein the one or more microcentrifuge tubes are rotated at a rotational speed of up to 60 rotations per minute (rpm).

7. The method of claim 1, wherein the one or more cell parameters comprise a cell radius, a nucleus radius, a membrane thickness, a cell wall permittivity, a cell membrane permittivity, a cytoplasm permittivity, a cell wall conductivity, a cell membrane conductivity, a cytoplasm conductivity, or a combination thereof.

8. The method of claim 1, wherein the one or more cell properties comprise a folding factor, a membrane capacitance, a membrane conductance, a cytoplasmic conductivity, or a combination thereof.

9. The method of claim 1, wherein the clinostat is a two-dimensional (2D) clinostat or a three-dimensional (3D) clinostat.

10. A clinostat, comprising:
a sample carriage configured to hold one or more sample tubes, the sample carriage supported by a shaft configured to rotate the sample carriage about a rotational axis;
a stepper motor communicatively coupled to the shaft, the stepper motor configured to rotate the sample carriage via the shaft to simulate a microgravity condition for the one or more sample tubes; and
stepper motor drive circuitry electrically coupled to the stepper motor, the stepper motor drive circuitry comprising a motor driver and controller configured to control rotational speed of the stepper motor during simulation of the microgravity condition.

11. The clinostat of claim 10, wherein the clinostat is a two-dimensional (2D) clinostat.

12. The clinostat of claim 11, comprising a support frame, wherein the sample carriage is directly coupled to the shaft, and the sample carriage supported between the support frame by the shaft.

13. The clinostat of claim 12, wherein the shaft is a stainless steel shaft.

14. The clinostat of claim 12, wherein the stepper motor is directly coupled to the shaft.

15. The clinostat of claim 10, wherein the sample carriage is fabricated from aluminum.

16. The clinostat of claim 10, wherein the clinostat is a three-dimensional (3D) clinostat configured to rotate about two rotational axes.

17. The clinostat of claim 16, comprising an inner frame rotationally coupled to an outer frame, wherein the inner frame secures the sample carriage at a rotational center of the clinostat and the outer frame is supported by the shaft, wherein the rotational axis is a first rotational axis and the outer frame rotates the sample carriage about the first rotational axis and the inner frame rotates the sample carriage about a second rotational axis.

18. The clinostat of claim 17, wherein the first and second rotational axes are substantially orthogonal.

19. The clinostat of claim 17, wherein the stepper motor is communicatively coupled to the shaft supporting the outer frame via a belt and pulley.

20. The clinostat of claim 17, comprising a second stepper motor communicatively coupled to the inner frame via a belt and pulley, the second stepper motor independently controlled from the other stepper motor.
